(19) United States
(12) Patent Application Publication
(10) Pub. No.: US 2019/0177774 A1
(43) Pub. Date: Jun. 13, 2019

(54) MOLECULAR REFERENCE CONTROLS

(71) Applicant: STRECK, INC., La Vista, NE (US)

(72) Inventors: Christopher Connelly, Greina, NE (US); Matthew R. Kreifels, Elkhorn, NE (US); Matthew Sobansky, Lincoln, NE (US)

(21) Appl. No.: 16/324,411

(22) PCT Filed: Aug. 11, 2017
(86) PCT No.: PCT/US17/46537

§ 371 Date: Feb. 8, 2019

Related U.S. Application Data
(60) Provisional application No. 62/514,471, filed on Jun. 2, 2017.
(60) Provisional application No. 62/374,192, filed on Aug. 12, 2016.

(51) Int. Cl. C12Q 1/689

(52) U.S. Cl. CPC C12Q 1/689; C12Q 2600/166

(57) ABSTRACT

Reference controls for use in downstream molecular technologies comprising a control composition including a sample base matrix, such as at least one biological fluid or biological fluid component, at least one condition component processed to be indicative of a condition, and at least one cross-linking agent.

### CLAIM OF PRIORITY

The present application claims the benefit of the filing dates of U.S. Provisional Application Nos. 62/374,192 filed on Aug. 12, 2016 and 62/514,471 filed on Jun. 2, 2017, which are incorporated by reference herein.

### FIELD

The present teachings relate to reference controls for use in downstream molecular technologies.

### BACKGROUND

Molecular diagnostics are a collection of techniques used to analyze biological markers in the genome and proteome. These markers are used to determine potential benefit from a specific therapy or risk of developing a certain disease or other health condition. For example, molecular diagnostic testing may include testing related to infectious diseases, cancer and inherited disease. Molecular diagnostics influence healthcare decisions and the diagnostic testing rate per person is growing. Thus, the prevalence of molecular testing and criticality of healthcare decisions made based upon the results of molecular diagnostic tests underscore the need to ensure that analytical results are reliable.

Molecular reference controls are a class of controls or standards used to verify or validate the performance of molecular diagnostic assays. Molecular diagnostic assays include, but are not limited to, enzyme-linked immunosorbent assay (ELISA), fluorescence in situ hybridization (FISH) and polymerase chain reaction (PCR). Molecular controls may be used as a comprehensive process control for various molecular instruments. For example, molecular controls are utilized in sample-to-answer platforms, real-time PCR instrumentation, digital PCR instrumentation, next-generation sequencers and upstream PCR target-enrichment strategies and microarray-based detection strategies. Molecular controls may serve as positive or negative controls for each downstream molecular detection methodology.

Examples of nucleic acid amplification controls include those in U.S. Pat. Nos. 9,127,048 and 9,127,049 and incorporated by reference herein. However, existing controls may be prepared in media that does not represent patient samples (e.g. “purified protein matrix” or buffer) rather than stabilized biological fluids or culture media. Existing controls kits may not be multiplexed, requiring the analysis of multiple separate samples. Existing control kits may not provide a negative control sample.

Several regulatory agencies have mandates that require clinical laboratories to utilize quality control materials. The International Organization of Standardization (ISO) provides certain criteria for quality control materials. “The laboratory shall use quality control materials that react to the examining system in a manner as close as possible to patient samples.” (ISO 15189) “Quality control materials shall be periodically examined with a frequency that is based on the stability of the procedure and the risk of harm to the patient from an erroneous result.” (ISO 15189) The College of American Pathologists (CAP) provides a molecular pathology checklist of requirements for molecular pathology laboratories. According to the CAP Molecular Pathology Checklist, “Controls are samples that act as surrogates for patient/client specimens. They are processed like a patient/client sample to monitor the ongoing performance of the entire analytic process in every run.” The Clinical and Laboratory Standards Institute (CLSI) also provides standards and guidelines for molecular reference controls.

Although there is a need for quality control samples in clinical/medical laboratories, many suppliers in the area of molecular diagnostics have failed to meet one or more regulations. These regulations include a product that is close as possible to patient samples, monitoring ongoing performance of the entire analytic process, testing control materials in the same manner as patient specimens and stability/storage of control materials. Thus, there is a need for molecular controls that mimic actual patient samples, are prepared and tested in the same manner as patient samples, provide accurate and reproducible results, and are stable over time to enable ongoing performance of the entire analytical system over time.

There is a need for process controls to verify platform operating accuracy for both sample processing and sample detection. Current molecular controls may not allow manufacturers or users of molecular technologies to test the entire process from extraction to detection. Furthermore, current molecular controls may not accurately mimic a clinical sample. An ideal control should have a matrix similar to that of the clinical sample thereby ensuring that it reacts to the examining instrument in a manner close as possible to the clinical sample. The use of non-human or non-animal origin components may result in less accurate results. There is a need for a stabilized cellular controls that accurately mimic a clinical sample.

Stabilization of molecular controls for downstream analysis requires consideration of multiple factors. The structural integrity and morphology of the cellular components needs to be retained. The amount of any relevant control material, such as control cell counts or pathogen counts needs to be controlled. The molecular control should provide enough nucleic acid and/or protein for the target of interest to control for the molecular assay method being used. The stabilization chemistry of the molecular control should stabilize any pathogen added to the control material, thus preventing any additional growth of the pathogen that would affect the controlled count and further provide a safety benefit during handling. The stabilization chemistry of the molecular control should stabilize any organic components of the sample base matrix. In addition, the stabilization chemistry must be compatible with nucleic acid and protein extraction technologies so the molecular control can be adequately analyzed by molecular detection platforms. For example, the stabilization chemistry must not reduce nucleic acid extraction efficiency as this would provide a result not consistent with an actual target copy number. The present teachings provide molecular reference controls with one or more of the aforementioned benefits.

The present teachings further provide molecular reference controls in which the pathogens or cellular targets of interest may be lab-engineered to contain specific nucleic acid targets of interest relevant to the type of control being developed. In one example, a cellular control for Gram-negative bacteria with multi-drug resistance may be developed, which includes transforming the bacterial species to contain the resistance mechanisms of interest, propagating the bacteria, and stabilizing the bacteria (e.g. inactivating and stopping growth) to include it with the control material. Therefore, the present teachings provide a stabilized cellular control that includes a cell line or pathogen with the stabilized target of interest to serve as a positive control for each downstream molecular detection methodology. The present teachings also provide a stabilized cellular control that includes biomolecules, including one or more of the following: cellular nucleic acids, cell-free nucleic acids and proteins.

### SUMMARY

The present teachings provide a control composition for a stabilized molecular control comprising a sample base matrix including one or more organic components and a cross-linking agent. The present teachings provide for a control composition for indicating positive presence of a condition comprising at least one biological fluid or biological fluid component, at least one condition component processed to be indicative of a condition, and at least one cross-linking agent.

The control composition may include an aldehyde and/or aldehyde donor agent. The at least one biological fluid may be blood. The at least one biological fluid may be selected from the group consisting of blood, serum, plasma, urine, fecal matter, saliva, sputum, cerebral spinal fluid, vaginal secretions, and semen. The at least one biological fluid may be blood culture or bacterial culture. The at least one biological fluid or biological fluid component may be of human origin. The at least one biological fluid or biological fluid component may be of animal origin.

The at least one biological fluid component may be selected from the group consisting of white blood cells, red blood cells, cell-free nucleic acids, proteins, fragmented nucleic acids, fragmented proteins, and any synthetic or engineered combination thereof. The at least one biological fluid component may be a nucleic acid or protein.

The at least one condition component indicative of a condition may be selected from the group consisting of inactivated viruses, attenuated viruses, plasm ids, bacteria, fungi, parasites, microbiota, engineered cell lines, wild-type cells and/or any microbiome combination thereof. The at least one biological fluid component may be an endogenously and/or exogenously modified component to indicate one or more of mutation, glycosylation, methylation, acetylation, ubiquitination, S-Nitrosylation, lipidation, GPI anchors, myristoylation, palmitoylation, prenylation, and phosphorylation.

The at least one condition component may be an inactivated pathogen. The at least one condition component may be an inactivated virus. The at least one condition component may be bacteria. The at least one condition component may be at least one Gram-negative organism that contains at least one mechanism of drug or treatment resistance. The at least one condition component may be at least one Gram-positive organism that contains at least one mechanism of drug or treatment resistance. The at least one condition component may include an immortalized and/or primary cell line containing a mutant phenotype or other molecular signature of interest.

The control composition may serve as an external control. The control composition may serve as an internal control. The control composition may serve as an internal control and the at least one biological fluid component may be a nucleic acid that is a control target of interest.

The at least one biological fluid or biological fluid component may be treated to mimic effects of a condition. The at least one biological fluid or biological fluid component may be substantially free of any treatment to mimic effects of a condition.

The control composition may be stable at room temperature. The control composition may be substantially free of any time-dependent component degradation when stored at room temperature. The control composition may be substantially free of any time-dependent component degradation when stored at 4° C. The control composition may be substantially free of any time-dependent component degradation when stored at 37° C.

The at least one cross-linking agent may include an aldehyde with one or more reactive groups. The at least one cross-linking agent may include formaldehyde, glutaraldehyde, phthaldehyde or a combination thereof. The at least one cross-linking agent may include N-Hydroxysuccinimide (NHS) ester functional group(s). The at least one cross-linking agent may include disuccinimidyl suberate and/or bis[sulfosuccinimidyl] suberate. The at least one cross-linking agent may include maleimide functional group(s). The at least one cross-linking agent may include dithiobismaleimidoethane (DTME) and/or bismaleimidohexane (BMH). The at least one cross-linking agent may include one or more functional groups of the following: haloacetyls, imidoesters, pyridyl disulfides, hydrazides, alkoxyamines, and carboiimides. The at least one cross-linking agent may be homobifunctional. The at least one cross-linking agent may be heterobifunctional.

The control composition may include a formaldehyde donor agent including imidazolidinyl urea (IDU), diazolidinyl urea (DU) or a combination thereof. The control composition may include chemical analytes and/or metabolites. The control composition may include one or more surfactants. The control composition may include one or more metabolic inhibitors. The control composition may include one or more nuclease inhibitors. The control composition may include one or more organic components.

The control composition may be utilized for one or more of the following: nucleic acid/protein purification protocols, sample-to-answer testing platforms, polymerase chain reaction protocols, fluorescence in situ hybridization (FISH) testing, genetic sequencing, flow cytometry, enzymatic testing, mass spectrometry or enzyme-linked immunosorbent assay (ELISA) testing.

The control composition may include a stabilization period of about 15 minutes to about 120 hours. The control composition may include a stabilization period of about 15 minutes. The control composition may be stable at room temperature. The control composition may be stable for at least up to 180 days at one or more of the following: 4° C., room temperature and 37° C.

### BRIEF DESCRIPTION OF THE DRAWINGS

FIG. 1 depicts graphs illustrating the threshold cycle (Ct) of chemically stabilized controls including K. pneumoniae containing a NDM gene and K. pneumoniae containing an OXA-48 gene.

FIG. 2 depicts a graph illustrating the linear regression of the logarithm of K. pneumoniae concentration vs. determined threshold cycle for NDM and OXA-48 resistance mechanisms on a testing platform.

FIG. 3 depicts graphs illustrating the threshold cycle (Ct) of chemically stabilized controls including K. pneumoniae containing a NDM gene and K. pneumoniae containing an OXA-48 gene in which at the 120 day time point, aliquots of the controls were transferred to storage at room temperature and 37° C. and analyzed following 7, 31, 45, and 62 days of storage.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The present teachings provide for a control composition for indicating positive presence of a condition comprising at least one biological fluid or biological fluid component, at least one condition component processed to be indicative of a condition, and at least one cross-linking agent. The control composition may serve as an external control. The control composition may serve as an internal control.

The at least one biological fluid may be selected from the group consisting of blood, serum, plasma, urine, fecal matter, saliva, sputum, cerebral spinal fluid, vaginal secretions, and semen. The at least one biological fluid may be blood. The at least one biological fluid may be blood culture or bacterial culture. The at least one biological fluid or biological fluid component may be of human origin. The at least one biological fluid or biological fluid component may be of animal origin.

The present teachings provide molecular control products that mimic patient samples and monitor the accuracy and precision of the entire analytical process. For example, cells containing specific molecular targets may be stabilized and used within a molecular control product. The stabilization process may be “tuned” to target inactivation at specific sites. The stabilization is “portable” and can be applied to numerous cell types and used in multiple assay types.

The molecular controls of the present teachings may utilize one or more agents to stabilize cells within biological matrices, enabling accurate and precise detection of nucleic acid and/or protein during performance of molecular tests. The one or more agents may include chemical stabilizers. The stabilizers may cross-link nucleic acids and/or proteins. The nucleic acids and/or proteins may be circulating, on the cell surface, or within the cell depending on the specific agent utilized.

The present teachings provide a stabilized cellular control that includes a cell line or pathogen with the stabilized target of interest to serve as a positive control for each downstream molecular detection methodology. The control composition of the present teachings includes stabilized components. The stabilized components may include any of the following: red blood cells, white blood cells, cell-free nucleic acid, fragmented nucleic acid, proteins, fragmented proteins, plasmids, inactivated/attenuated virus, fungi, parasites, microbiome components, engineered cell lines and wild-type cell lines. For example, the fragments may range in size from 5 bp to 3500 bp. The stabilized components may include any synthetic or engineered combination thereof of the foregoing. The control composition may include chemical analytes and/or metabolites. The control composition may be indicative of reference values for chemical analytes and/or metabolites.

The control composition of the present teachings is compatible with different methodologies. The methodologies may include nucleic acid/protein purification protocols, sample-to-answer platforms, polymerase chain reaction based techniques, fluorescence in situ hybridization (FISH), next-generation sequencing (NGS), flow cytometric applications, enzyme-linked immunosorbent assay (ELISA) and enzymatic tests. The design of the control composition depends largely on the base sample matrix. For example, a blood control may contain but is not limited to the following: red blood cells, white blood cells, relevant pathogens or pathogen cells and stabilization component.

The present teachings may provide reference controls for use in downstream molecular technologies comprising a control composition including a sample base matrix, such as at least one biological fluid or biological fluid component, at least one condition component processed to be indicative of a condition, and a cross-linking agent.

The present teachings provide a control composition for indicating positive presence of a condition. The control composition may include at least one biological fluid component, growth media (e.g. bacterial or blood culture media), or other clinically relevant solution. The growth media may be solid, liquid or semi-solid. The control composition may include at least one condition component processed to be indicative of a condition. The control composition may include an aldehyde or aldehyde donor agent. In one example, the control composition comprises at least one human or animal-origin blood component processed to resemble a human blood component; at least one condition component processed to be indicative of a condition; and a cross-linking agent.

The control composition may be stored at room temperature or cold storage. The control composition may be substantially free of any time-dependent component degradation when stored at room temperature. The control composition may be substantially free of any time-dependent component degradation when stored at 4° C. The control composition may be substantially free of any time-dependent component degradation when stored at 37° C. The control composition may provide long term stability. For example, the control composition may be stable for at least six months. The control composition may be stable for at least one year. The control composition may be stable for at least two years. The control composition may be stable for at least three years.

The control may be an external control. The control may be an internal control. The control may be both an external and an internal control. The control composition may be utilized for one or more of the following: nucleic acid/protein purification protocols, sample-to-answer testing platforms, polymerase chain reaction protocols, fluorescence in situ hybridization (FISH) testing, genetic sequencing, flow cytometry, enzymatic testing, mass spectrometry or enzyme-linked immunosorbent assay (ELISA) testing. The control composition provides an accurate control for the isolation of nucleic acid. The control composition may be used with many different types of isolation technologies including spin columns, microfluidics and magnetic beads. The control composition may be tested directly without extraction.

The control composition may include actual blood, serum, plasma, urine, fecal matter, saliva, sputum, cerebral spinal fluid, vaginal secretions, semen or any other suitable biological or non-biologic matrix. The control composition may include blood culture. The control composition may include bacterial culture.

The biological fluid component may be selected from the group consisting of white blood cells, red blood cells, cell-free nucleic acids, proteins, fragmented nucleic acids, fragmented proteins, and any synthetic or engineered combination thereof. The biological fluid or biological fluid component may be of human and/or animal origin. The control composition may include at least one human biological fluid component or animal-origin biological fluid component processed to resemble a human biological fluid component.

The control composition may contain exogenous or endogenous modifications to native or non-native molecules that mimic biological phenomenon indicative of disease or non-disease status. The modifications may include, but are but not limited to mutation, glycosylation, methylation, acetylation, ubiquitination, S-Nitrosylation, lipidation, GPI anchors, myristoylation, palm itoylation, prenylation, or phosphorylation. The at least one biological fluid component may be an endogenously and/or exogenously modified component to indicate one or more of mutation, glycosylation, methylation, acetylation, ubiquitination, S-Nitrosylation, lipidation, GPI anchors, myristoylation, palmitoylation, prenylation, and phosphorylation.

The at least one biological fluid or biological fluid component may be treated to mimic effects of a condition. The at least one biological fluid or biological fluid component may be substantially free of any treatment to mimic effects of a condition. The biological fluid component may be a nucleic acid. The biological fluid component may be a protein. In one example, the control composition serves as an internal control and the at least one biological fluid component is a nucleic acid that is a control target of interest. The nucleic acid may be of human origin.

The control composition may include at least one condition component processed to be indicative of a condition. The condition component may be indicative of a non-disease state. The condition component may be indicative of a disease state. For example, the condition component may be indicative of an infection, an autoimmune condition, chromosomal abnormalities or a malignancy such as a tumor. The condition component indicative of a condition may be selected from the group consisting of inactivated viruses, attenuated viruses, plasmids, bacteria, fungi, parasites, microbiota, engineered cell lines, wild-type cells and/or any microbiome combination thereof.

The condition component may be an inactivated pathogen. The present teachings may expedite the time required to stabilize and inactivate the pathogen. The control composition may include a stabilization period of about 15 minutes to about 120 hours. For example, the control composition may include a stabilization period of about 15 minutes. The pathogen may be inactivated in less than fifteen minutes upon contact with the one more components of the control composition. The pathogen may be inactivated in about fifteen minutes upon contact with the one more components of the control composition. The pathogen may be inactivated in about one hour or less upon contact with the one more components of the control composition. The pathogen may be inactivated in about two hours upon contact with the one more components of the control composition. The pathogen may be inactivated in about six hours upon contact with the one more components of the control composition. The pathogen may be inactivated in about two to about six hours upon contact with the one or more components of the control composition. The pathogen may be inactivated in about two to about twelve hours upon contact with the one more components of the control composition. The pathogen may be inactivated in about twenty-four hours upon contact with the one more components of the control composition. The pathogen may be inactivated in about 5 days or less upon contact with the one more components of the control composition.

The condition component may be an inactivated virus. The condition component may be bacteria. The condition component may be at least one Gram-negative organism that contains at least one mechanism of drug or treatment resistance (e.g. antibiotic resistance). The condition component may be at least one Gram-positive organism that contains at least one mechanism of drug or treatment resistance (e.g. antibiotic resistance). The condition component may include a cell line containing a mutant phenotype or other molecular signature of interest. The cell line may be an immortalized and/or primary cell line. The condition component may include an immortalized and/or primary cell line containing a mutant phenotype or other molecular signature of interest.

In one example, the sample matrix is a blood component and the at least one condition component is an inactivated virus. The virus may be inactivated in about fifteen minutes to about twelve hours upon contact with the one more components of the control composition. In one example, the sample matrix is blood-based or relevant body fluid based and the at least one condition component includes immortalized cell lines. The stabilized molecular control provides a control for circulating tumor cells relevant to a given cancer type. Thus, providing a DNA, RNA or protein source for multiple molecular technologies, including PCR, next-generation sequencing and RNA sequencing.

In one example, the molecular reference controls may include controls that provide for the stabilization of bacterial cells and subsequent PCR analysis of DNA targets within these cells. The bacterial cells may contain KPC, NDM, OXA-48, CTX-M-15, and/or other relevant resistance mechanisms. The bacteria may be Gram-negative bacteria such as Escherichia coli (E. coli), Neisseria sicca (N. sicca) and Klebsiella pneumoniae (K. pneumoniae). The bacteria may be Gram-positive bacteria such as Corynebacterium pseudodiphtheriticum (C. pseudo).

Stabilization may occur via the use of chemical stabilizers that inactivate cells by crosslinking biomolecules containing reactive functional groups. The chemical stabilizer may be an aldehyde. The aldehyde may include one or more functional groups. For example, the aldehyde may be one of the following: formaldehyde, glutaraldehyde, o-phthalaldehyde, and trimesaldehyde. For example, the aldehyde may be selected from the group consisting of paraformaldehyde, formaldehyde, acetaldehyde, propionaldehyde, n-butyraldehyde, benzaldehyde, p-nitrobenzaldehyde, p-tolualdehyde, salicylaldehyde, phenylacetaldehyde. The chemical stabilizer may be an N-Hydroxysuccinimide (NHS) ester. For example, the NHS ester may be selected from the following: Disuccinimidyl Suberate (DSS), Bis[sulfosuccinimidyl] Suberate (BS3), Dithiobis (succinim idyl propionate) (DSP), and 3,3′-Dithiobis(sulfosuccinimidyl propionate) (DTSSP). The NHS ester may provide internal crosslinking, for example, via Disuccinimidyl Suberate (DSS). The NHS ester may provide external crosslinking, for example, via Bis[sulfosuccinimidyl] Suberate (BS3). The chemical stabilizer may include one or more of the following: formaldehyde, glutaraldehyde, o-phthalaldehyde, disuccinim idyl suberate, and bis[sulfosuccinimidyl] suberate.

In one example, bacterial cells containing target gene sequences were stabilized with various cross-linkers and evaluated by microbiological methods. Viability and sterility were evaluated at various time points following addition of stabilizer. Time to stabilization was evaluated for particular bacteria/stabilizer combinations as shown below in Table 1.

TABLE 1

| | | Time to | |
|---|---|---|---|
| Bacteria | Stabilizer | Stabilization | |
| E. coli | Formaldehyde | 24 | hours |
| | Glutaraldehyde | 2 | hours |
| | o-Phthalaldehyde | 15 | minutes |
| | Disuccinimidyl Suberate | 5 | days |
| | Bis[sulfosuccinimidyl] | >5 | days |
| | Suberate | | |
| N. sicca | Formaldehyde | 24 | hours |
| | Glutaraldehyde | 2 | hours |
| | o-Phthalaldehyde | 2 | hours |
| C. psuedo | Formaldehyde | 24 | hours |
| | Glutaraldehyde | 24 | hours |
| | o-Phthalaldehyde | 24 | hours |

Cross-linking agents may react with specific functional groups on proteins. For example, the cross-linkers may react with any of the following functional groups: primary amines, carboxyls, sulfhydryls, and carbonyls. The cross-linkers may react with amines. The cross-linkers may be specific for primary amine groups, such as genipin. The cross-linkers may interact with sulhydryls groups, such as maleimides and haloacetyls. The cross-linking agent may be a maleimide crosslinker which targets sulfhydryl functional groups, such as dithiobismaleimidoethane (DTME) or bismaleimidohexane (BMH). The cross-linking agent may include any of the following: aldehydes, NHS esters, maleimides, haloacetyls, imidoesters, pyridyl disulfides, hydrazides, alkoxyamines, and carboiimides. The cross-linking agent may be homobifunctional or heterobifunctional.

The at least one cross-linking agent may include formaldehyde, glutaraldehyde, phthaldehyde or a combination thereof. The at least one cross-linking agent may include N-Hydroxysuccinimide (NHS) ester functional group(s). The at least one cross-linking agent may include disuccinimidyl suberate and/or bis[sulfosuccinimidyl] suberate. The at least one cross-linking agent may include maleimide functional group(s). The at least one cross-linking agent may include dithiobismaleimidoethane (DTME) and/or bismaleimidohexane (BMH). The at least one cross-linking agent may include one or more functional groups of the following: haloacetyls, imidoesters, pyridyl disulfides, hydrazides, alkoxyamines, and carboiimides.

The chemical stabilizer may include a molecular weight of about 25 g/mol to about 575 g/mol. The chemical stabilizer may include a molecular weight of about 25 g/mol to about 150 g/mol. The chemical stabilizer may include a molecular weight of about 350 g/mol to about 575 g/mol.

The chemical stabilizer may be non-polar (hydrophobic). The chemical stabilizer may be polar (hydrophilic). The chemical stabilizer may be amphipathic (have both hydrophilic and hydrophobic properties).

The log P value of the chemical stabilizer may be from about −3.00 to about 3.00. The log P value of the chemical stabilizer may be from about −2.00 to about 2.00. The log P value of the chemical stabilizer may be from about −1.00 to about 1.00. The log P value of the chemical stabilizer may be from about −0.50 to about 0.50.

The spacer arm length or the molecular span of a crosslinker (i.e., the distance between conjugated molecules), may vary. For example, the spacer arm length may be from about 2 to about 12 angstroms. The spacer arm length may be about 2 Å. The spacer arm length may be about 3 Å. The spacer arm length may be about 10 Å. The spacer arm length may be about 11 Å.

The control composition may be formaldehyde free. The control composition may include formaldehyde. The control composition may include a formaldehyde donor agent. The formaldehyde donor agent may release formaldehyde upon contact with an aqueous substance.

Formaldehyde donor agents that may be used include, but are not limited to, diazolidinyl urea (DU), imidazolidinyl urea (IDU), dimethylol urea, 2-bromo-2-nitropropane-1,3-diol, 5-hydroxymethoxymethyl-1-aza-3,7-dioxabicyclo (3.3.0)octane and 5-hydroxymethyl-1-aza-3,7-dioxabicyclo (3.3.0)octane and 5-hydroxypoly [methyleneoxy]methyl-1-aza-3,7-dioxabicyclo (3.3.0)octane, bicyclic oxazolidines, DMDM hydantoin, sodium hydroxymethylglycinate, hexamethylenetetramine chloroallyl chloride, biocides, a water-soluble zinc salt or any combination thereof.

The formaldehyde donor agent may include a compound that may release formaldehyde with an electron deficient functional group. The control composition may include a compound that includes at least one functional group capable of reacting with an electron deficient functional group of formaldehyde. For example, an amine compound that reacts with formaldehyde to form methylol or imine Schiff base or a cis-diol compound that reacts with formaldehyde to form a cyclic acetal. For example, the control composition may include glycine, Tris(hydroxymethyl)aminomethane (TRIS), urea, allantoin, sulfites or any combination thereof.

The control composition may include one or more of the following: amino acids, alkyl amines, polyamines, primary amines, secondary amines, ammonium salts, or a combination thereof. The control composition may include one or more of the following: glycine, lysine, ethylene diamine, arginine, urea, adenine, guanine, cytosine, thymine, sperm idine, or any combination thereof.

The composition may include any suitable stabilizing agent. The control composition may include a cross-linking agent. The cross-linking agent may include include an aldehyde with one or more reactive groups. The control composition may include an aldehyde and/or aldehyde donor agent. The aldehyde may be selected from the group consisting of: formaldehyde, glutaraldehyde, phthaldehyde or a combination thereof. The control composition may include a heterocyclic urea. The heterocyclic urea may be selected from the group consisting of: diazolidinyl urea (DU), imidazolidinyl urea (IDU) or a combination thereof. The stabilizing agent may be selected from the group consisting of: imidazolidinyl urea (IDU), one or more biocides (e.g. Nuosept 145, Nuosept 95), formaldehyde, glutaraldehyde, phthaldehyde and any combination thereof. The control composition may include an aldehyde, an alcohol, a heterocyclic urea or a mixture thereof.

The stabilizing agent may be present in an amount of about 0.000001% to about 10%. The stabilizing agent may be present in an amount of about 0.000001% to about 25%. The stabilizing agent may be present in an amount of about 0.00001% to about 50%. The stabilizing agent may be removed following stabilization. Thus, the final stabilizing concentration in a control sample may be non-detectable or substantially equivalent to zero.

In one example, the control composition includes imidazolidinyl urea (IDU). The concentration ranges of imidazolidinyl urea (IDU) may be from about 0.0001% to about 10%. The concentration of imidazolidinyl urea (IDU) may be about 1%. The concentration of imidazolidinyl urea (IDU) may be about 2.5%. The concentration of imidazolidinyl urea (IDU) may be about 5%.

In one example, the control composition includes [[[(2-dihydro-5-methyl-3(2H)-oxazolyl)-1-methylethoxy]methoxy]methoxy]methanol (e.g., Nuosept 145). The concentration range of [[[(2-dihydro-5-methyl-3(2H)-oxazolyl)-1-methylethoxy]methoxy]methoxy]methanol may be from about 0.0001% to about 50%. The concentration range of [[[(2-dihydro-5-methyl-3(2H)-oxazolyl)-1-methylethoxy]methoxy]methoxy]methanol may be from about 0.0001% to about 25%. The concentration of [[[(2-dihydro-5-methyl-3(2H)-oxazolyl)-1-methylethoxy]methoxy]methoxy]methanol may be about 1%. The concentration of [[[(2-dihydro-5-methyl-3(2H)-oxazolyl)-1-methylethoxy]methoxy]methoxy]methanol may be about 2.5%. The concentration of [[[(2-dihydro-5-methyl-3(2H)-oxazolyl)-1-methylethoxy]methoxy]methoxy]methanol may be about 5%. The concentration of [[[(2-dihydro-5-methyl-3(2H)-oxazolyl)-1-methylethoxy]methoxy]methoxy]methanol may be about 10%. The concentration of [[[(2-dihydro-5-methyl-3(2H)-oxazolyl)-1-methylethoxy]methoxy]methoxy]methanol may be about 20%.

In one example, the control composition includes a mixture of bicyclic oxazolidines (e.g. Nuosept 95). The concentration range of bicyclic oxazolidines may be from about 0.0001% to about 50%. The concentration range of bicyclic oxazolidines may be from about 0.0001% to about 25%. The concentration of bicyclic oxazolidines may be about 1%. The concentration of bicyclic oxazolidines may be about 2.5%. The concentration of bicyclic oxazolidines may be about 5%. The concentration of bicyclic oxazolidines may be about 10%. The concentration of bicyclic oxazolidines may be about 20%.

In one example, the control composition includes formaldehyde. The concentration range of formaldehyde may be from about 0.0001% to about 10%. The concentration of formaldehyde may be about 1%. The concentration of formaldehyde may be about 2.5%. The concentration of formaldehyde may be about 5%. The concentration of formaldehyde may be about 10%.

In one example, the control composition includes glutaraldehyde. The concentration range of glutaraldehyde may be from about 0.0001% to about 10%. The concentration of glutaraldehyde may be about 0.1%. The concentration of glutaraldehyde may be about 1%. The concentration of glutaraldehyde may be about 2.5%. The concentration of glutaraldehyde may be about 5%. The concentration of glutaraldehyde may be about 10%.

The control composition may include one or more surfactants. The surfactant may include polyethylene glycol. The surfactant may include variants of polyethylene glycol. The concentration of surfactant may be from about 0.5% to about 10%. The concentration of surfactant may be about 1%. The concentration of surfactant may be about 2.5%. The concentration of surfactant may be about 5%. The concentration of surfactant may be about 10%. The control composition may include one or more detergents. The detergent may be ionic, non-ionic, or zwitterionic. For example, the detergent may be selected from the Brij family of detergents. The concentration of detergent may be from about 0.001% to about 5%. The concentration of detergent may be about 0.5%. The concentration of detergent may be about 2.0%. The concentration of detergent may be about 5%.

The control composition may include one or more metabolic inhibitors. One or more metabolic inhibitors may be selected from the group consisting of: glyceraldehyde, dihydroxyacetone phosphate, glyceraldehyde 3-phosphate, 1,3-bisphosphoglycerate, 3-phosphoglycerate, 2-phosphoglycerate, phosphoenolpyruvate, pyruvate and glycerate dihydroxyacetate, sodium fluoride, K₂C₂0₄ and any combination thereof. In one example, the control composition includes glyceraldehyde. In one example, the control composition includes sodium fluoride. In one example, the control composition includes both glyceraldehyde and sodium fluoride.

The control composition may include one or more nuclease inhibitors. Nuclease inhibitors that may be used include, but are not limited to diethyl pyrocarbonate, ethanol, aurintricarboxylic acid (ATA), formamide, vanadyl-ribonucleoside complexes, macaloid, ethylenediamine tetraacetic acid (EDTA), proteinase K, heparin, hydroxylamine-oxygen-cupric ion, bentonite, ammonium sulfate, dithiothreitol (DTT), beta-mercaptoethanol, cysteine, dithioerythritol, tris (2-carboxyethyl) phosphene hydrochloride, or a divalent cation such as Mg⁺², Mn⁺², Zn⁺², Fe⁺², Ca⁺², Cu⁺² and any combination thereof. In one example, the control composition includes ethylenediamine tetraacetic acid (EDTA). In one example, the control composition includes aurintricarboxylic acid (ATA). In one example, the control composition includes both ethylenediamine tetraacetic acid (EDTA) and aurintricarboxylic acid (ATA).

The control composition may include one or more protease inhibitors. Protease inhibitors that may be used include, but are not limited to antipain, aprotinin, chymostatin, elastatinal, phenylmethylsulfonyl fluoride (PMSF), APMSF, TLCK, TPCK, leupeptin, soybean trypsin inhibitor, indoleacetic acid (IAA), E-64, pepstatin, VdLPFFVdL, EDTA, 1,10-phenanthroline, phosphoramodon, amastatin, bestatin, diprotin A, diprotin B, alpha-2-macroglobulin, lima bean trypsin inhibitor, pancreatic protease inhibitor, egg white ovostatin egg white cystatin, and any combination thereof.

The control composition may include one or more enzyme inhibitors. Enzyme inhibitors that may be used include, but are not limited to diethyl pyrocarbonate, ethanol, aurintricarboxylic acid (ATA), glyceraldehydes, sodium fluoride, ethylenediaminetetraacetic acid (EDTA), form am ide, vanadyl-ribonucleoside complexes, macaloid, heparin, hydroxylamine-oxygen-cupric ion, bentonite, ammonium sulfate, dithiothreitol (DTT), beta-mercaptoethanol, cysteine, dithioerythritol, tris (2carboxyethyl) phosphene hydrochloride, a divalent cation such as Mg+2, Mn+2, Zn+2, Fe+2, Ca+2, Cu+2 and any combination thereof. The control composition may include one or more enzyme activators. The control composition may include one or more compounds that control cell cycle, cell death and other cellular processes.

It should be appreciated that the above specific listings of compounds may contain a measure of overlap, which recognizes the sometimes-overlapping function of certain specific compounds. One of skill in the art should understand and appreciate this aspect of the disclosure.

The stabilization process preserves cells to allow further processing. The stabilization process maintains the architecture and morphology of the initial sample, prevents decomposition and lysis of cells, eliminates cell replication to maintain known concentrations, and allows for use of the cells in downstream application. For example, the stabilized bacteria are suitable for extraction and use in qPCR. The qPCR reaction may be operated in singleplex or multiplex. The multiplex reaction may include genetic targets for bacterial infection and associated antibiotic resistance. For example, the multiplex mix may include genetic targets from E. coli, K. pneumoniae, C. pseudo and N. sicca.

The present teachings provide a control including cells stabilized through cross-linking reactions. This stabilization allows the cells to be incorporated into biological matrices and stored for extended time periods at refrigerated or ambient conditions. However, the cross-linking is not so extensive as to result in reactions with nucleic acids or prevent extraction during typical DNA/RNA/protein extraction procedures employed in the performance of real-time PCR testing and other molecular testing methodologies.

The molecular reference controls of the present teachings may be complete cellular controls, compatible with molecular detection platforms, and in multiplex format. The molecular reference controls of the present teachings may include control matrices with clinically-relevant genetic targets for bacterial infection and associated antibiotic resistance. For example, the molecular reference controls may include stabilized components for each relevant sample matrix and Escherichia coli or Klebsiella pneumoniae cells containing KPC, NDM, OXA-48, CTX-M-15, and/or other relevant resistance mechanisms. For example, the molecular reference controls of the present teachings may be utilized in testing related to milk, blood, feces, urine, bacterial culture, positive blood culture, bacterial suspension or rectal/fecal swab. For example, the molecular reference controls may be utilized with Streck ARM-D® Kits, ampC and β-Lactamase (Streck Inc., Omaha, Nebr.), BioFire FilmArray® BCID Panel (BioFire Diagnostics LLC, Salt Lake City, Utah), and Cepheid® GeneXpert® Carba-R tests and MRSA panel (Cepheid, Sunnyvale, Calif.), and Luminex (Nanosphere) Verigene® Bloodstream Infection Tests (Nanosphere, Northbrook, Ill.). The molecular reference controls may be utilized for other diagnostic tests.

Thus, the molecular controls of the present teachings may provide a stable patient-like process control for use with molecular diagnostic tests, including those that are used in the detection of antimicrobial resistance genes. These controls are designed to mimic patient samples in appearance, composition, and performance. Furthermore, they may be used to monitor the accuracy and precision of the entire system including lysis, nucleic acid isolation, amplification, detection, and data computation.

The control composition may be stable for about 2 weeks to about 2 years at one or more of the following: 4° C., room temperature and 37° C. The control composition may be stable for at least up to 30 days at one or more of the following: 4° C., room temperature and 37° C. The control composition may be stable for at least up to 60 days at one or more of the following: 4° C., room temperature and 37° C. The control composition may be stable for at least up to 90 days at one or more of the following: 4° C., room temperature and 37° C. The control composition may be stable for at least up to 180 days at one or more of the following: 4° C., room temperature and 37° C. The control composition may be stable for at least up to 270 days at one or more of the following: 4° C., room temperature and 37° C. The control composition may be stable for at least up to one year at one or more of the following: 4° C., room temperature and 37° C. The control composition may be stable for at least up to two years at one or more of the following: 4° C., room temperature and 37° C.

The molecular tests of the present teachings may provide equivalent analytical performance when held for at least 7 days at a temperature from about 4° C. to about 37° C. The molecular tests of the present teachings may provide equivalent analytical performance when held for up to at least 180 days at a temperature about 4° C. to about 37° C. The molecular tests of the present teachings may provide equivalent analytical performance when held for up to at least 365 days at a temperature about 4° C. to about 37° C. For example, the molecular tests of the present teachings may provide equivalent analytical performance when held for about 7, 14, 30, 45, 90, 150, 270, or 365 days at 4° C. For example, the molecular tests of the present teachings may provide equivalent analytical performance when held for about 15 days at room temperature (e.g. 18.3° C.-26.7° C.). For example, the molecular tests of the present teachings may provide equivalent analytical performance when held for about 15 days at 37° C.

The control composition or compositions of the present teachings provide a control which maintains the appearance of a fresh sample and are stable. The control composition may have an open vial stability of at least about six weeks. The control composition may have an open vial stability of at least about three months. The control composition may have an open vial stability of at least about six months. The control composition may have an open vial stability of at least about 12 months. The control composition may have a closed vial stability of at least about six months. The control composition may have a closed vial stability of at least about 12 months. The control composition may have a closed vial stability of at least about 2 years. The control composition of the present teachings provide a stable controls suitable for use with nucleic acid/protein extraction and molecular detection platforms.

### EXAMPLES

The present teachings provide full process controls for use with molecular diagnostic methods. These controls may be used with tests for bacterial identification and genotypic detection of antibiotic resistance. Stability studies of the control samples of the present teachings were conducted in the following examples. Control samples were prepared to stabilize the components for each relevant sample matrix and Escherichia coli or Klebsiella pneumoniae cells containing KPC, NDM, OXA48, CTX-M-15, and/or other relevant resistance mechanisms. Stability of the prepared controls was monitored by comparing initial results to those obtained after storage. Tests evaluated include the Streck ARM-D® Kits, ampC and β-Lactamase (bacterial culture), BioFire FilmArray® BCID Panel (positive blood culture), and Cepheid® GeneXpert® Carba-R test (bacterial suspension or rectal swab). The results indicate that molecular controls, positive for each target, demonstrate the expected identification profile of organisms and/or resistance mechanisms using the Streck ARM-D Kits, ampC and β-Lactamase, BioFire FilmArray BCID, and Cepheid GeneXpert Carba-R tests. Stability analysis demonstrates equivalent analytical performance when controls are held for up to 180 days at 4° C.

### Example 1

Prepared controls of the present teachings were evaluated with Streck ARM-D® Kits, β-Lactamase (bacterial culture). Klebsiella pneumoniae containing NDM, OXA-48, and CTX-M-15 resistance genes were chemically stabilized. Stabilized cells were prepared as a bacterial culture by diluting stabilized cells to 1×10⁸ cells/mL in Luria-Bertani broth (Gibco®). An aliquot of each prepared sample was stored at 4° C., room temperature, and 37° C. Samples were extracted from 200 μL of bacterial culture at each time point using the QuickGene DNA tissue kit S and the QuickGene-810 system. DNA extracts were examined by real-time PCR (qPCR) using the QuantStudio 7 Flex Real-Time PCR System (Applied Biosystems®).

Table 2 depicts the threshold cycle values determined for a control sample prepared with stabilized Klebsiella pneumoniae containing a KPC resistance gene. Table 3 depicts the threshold cycle values determined for a control sample prepared with stabilized Klebsiella pneumoniae containing the CTX-M-15, OXA-48, and NDM resistance genes. The control samples were analyzed using the Streck ARM-D Kit, β-Lactamase over the course of 45 days.

TABLE 2

| | KPC | | |
|---|---|---|---|
| Day | 4° C. | RT | 37° C. |
| 0 | 20.0 | 19.8 | 19.7 |
| 1 | 19.9 | 19.0 | 19.8 |
| 7 | 19.5 | 19.7 | 19.3 |
| 15 | 20.7 | 20.4 | 19.5 |
| 45 | 19.4 | 19.5 | 19.6 |
| Mean | 19.9 ± 0.5 | 19.7 ± 0.5 | 19.6 ± 0.2 |
| Δ | −0.6 | −0.3 | 0.0 |

Note - Results corrected for DNA concentration measured using Qubit dsDNA HS Assay.

Initial real-time PCR results of a stabilized Klebsiella pneumoniae containing the KPC resistance gene indicated positive results for KPC. Real-time PCR results of a stabilized Klebsiella pneumoniae containing the KPC resistance gene following 45 days storage at 37° C. indicated positive results for KPC. The above results demonstrate the suitability of the prepared control when used with the Streck ARM-D Kit, β-Lactamase. This suitability is exemplified by stability of the prepared bacterial cultures over 45 days when held at 4° C., room temperature, or 37° C.

TABLE 3

| | CTX-M-15 | | | OXA-48 | | | NDM | | |
|---|---|---|---|---|---|---|---|---|---|
| Day | 4° C. | RT | 37° C. | 4° C. | RT | 37° C. | 4° C. | RT | 37° C. |
| 0 | 22.1 | 22.0 | 21.9 | 20.5 | 20.3 | 20.2 | 22.5 | 22.5 | 22.6 |
| 1 | 21.9 | 22.2 | 21.5 | 20.2 | 20.1 | 20.2 | 22.7 | 22.8 | 22.3 |
| 7 | 21.7 | 21.6 | 21.3 | 20.2 | 21.1 | 19.7 | 21.4 | 22.2 | 21.6 |
| 15 | 24.0 | 23.5 | 21.7 | 21.8 | 20.8 | 19.5 | 21.2 | 22.2 | 21.9 |
| 45 | 21.2 | 21.8 | 21.3 | 20.7 | 20.5 | 20.2 | 21.8 | 21.5 | 21.1 |
| Mean | 22.2 ± 1.1 | 22.2 ± 0.8 | 21.5 ± 0.3 | 20.7 ± 0.8 | 20.5 ± 0.5 | 19.9 ± 0.4 | 21.9 ± 0.8 | 22.2 ± 0.3 | 21.9 ± 0.4 |
| Δ | −0.9 | −0.2 | −0.6 | 0.2 | 0.2 | −0.1 | −0.7 | −1.0 | −1.5 |

Note
Results corrected for DNA concentration measured using Qubit dsDNA HS Assay.

Initial real-time PCR results of a stabilized Klebsiella pneumoniae containing NDM, OXA-48, and CTX-M-15 resistance genes indicated positive results for NDM, OXA-48, and CTX-M-15 genes. Real-time PCR results of a stabilized Klebsiella pneumoniae containing NDM, OXA-48, and CTX-M-15 resistance genes following 45 days storage at 37° C. indicated positive results for NDM, OXA-48, and CTX-M-15 genes. The above results demonstrate the suitability of the prepared control when used with the Streck ARM-D Kit, β-Lactamase. This suitability is exemplified by stability of the prepared bacterial cultures over 45 days when held at 4° C., room temperature, or 37° C.

### Example 2

Prepared controls of the present teachings were evaluated with the Cepheid Xpert Carba-R kit. Klebsiella pneumoniae containing NDM, OXA-48, and CTX-M-15 resistance genes were chemically stabilized. Stabilized cells were prepared as 10-fold serial dilutions of a stock bacterial suspension of stabilized cells to working concentrations of 1×10⁸, 1×10⁷, 1×10⁶ or 1×10⁵ cells/mL in phosphate buffered saline, pH 7.4 (Gibco®). Each sample was stored at 4° C. At each designated time point, a 10 μL sample was analyzed. There was no significant shift from the mean for any of the detected genes as indicated by the determined threshold cycle for each control. The utility of these controls when used with the Cepheid Xpert Carba-R kit is shown by the stability of the prepared bacterial suspensions over 180 days when held at 4° C.

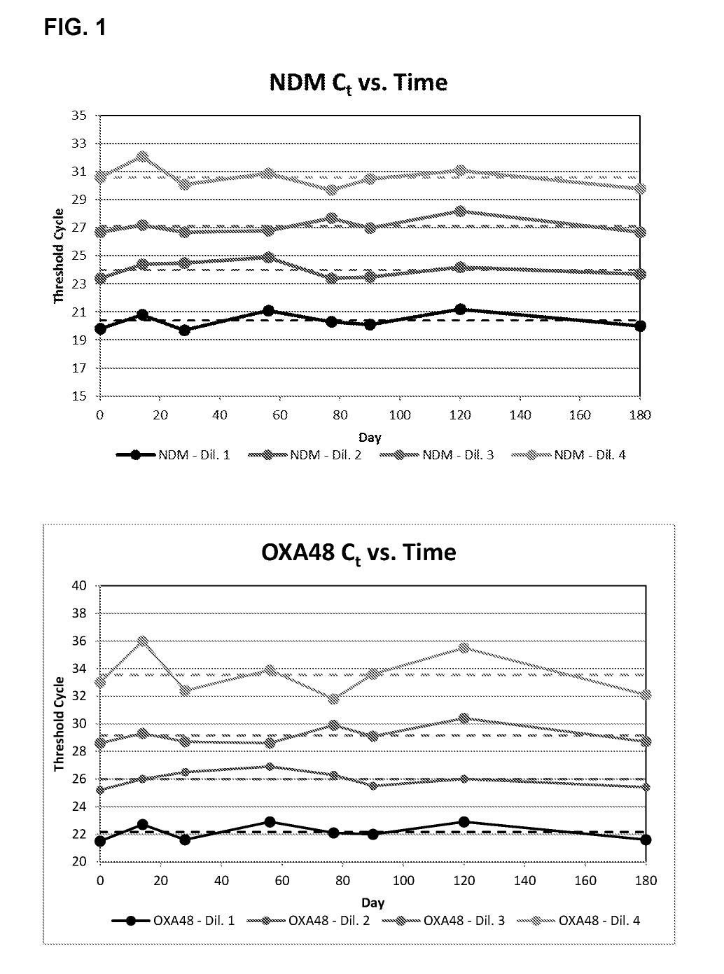

FIG. 1 illustrates that was no significant shift from the mean for the detected genes as indicated by the determined threshold cycle of a stabilized control including a NDM gene and a stabilized control including an OXA-48 gene. Determined threshold cycle of four suspensions of K. pneumoniae with NDM and OXA-48 genes at Dil. 1: 1×10⁸ cells/mL, Dil. 2: 1×10⁷ cells/mL, Dil. 3: 1×10⁸ cells/mL, and Dil. 4: 1×10⁸ cells/mL are depicted. The mean value of all measurements at a given concentration is represented by the dashed line.

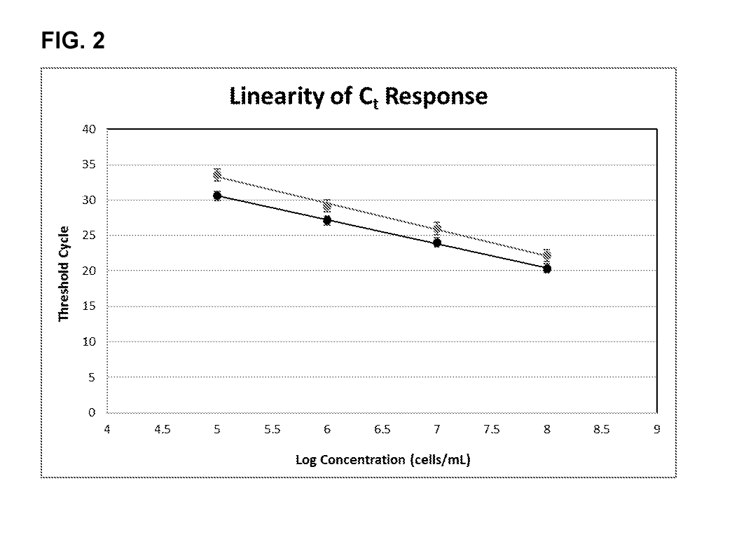

Additionally, linearity of the Ct response is maintained across the working concentration range as depicted in FIG. 2. FIG. 2 depicts the linear regression of the logarithm of K. pneumoniae concentration vs. determined threshold cycle on the Cepheid Xpert Carba-R test. The slope of the best fit line for the NDM and OXA-48 resistance mechanisms are −3.40 and −3.77, respectively.

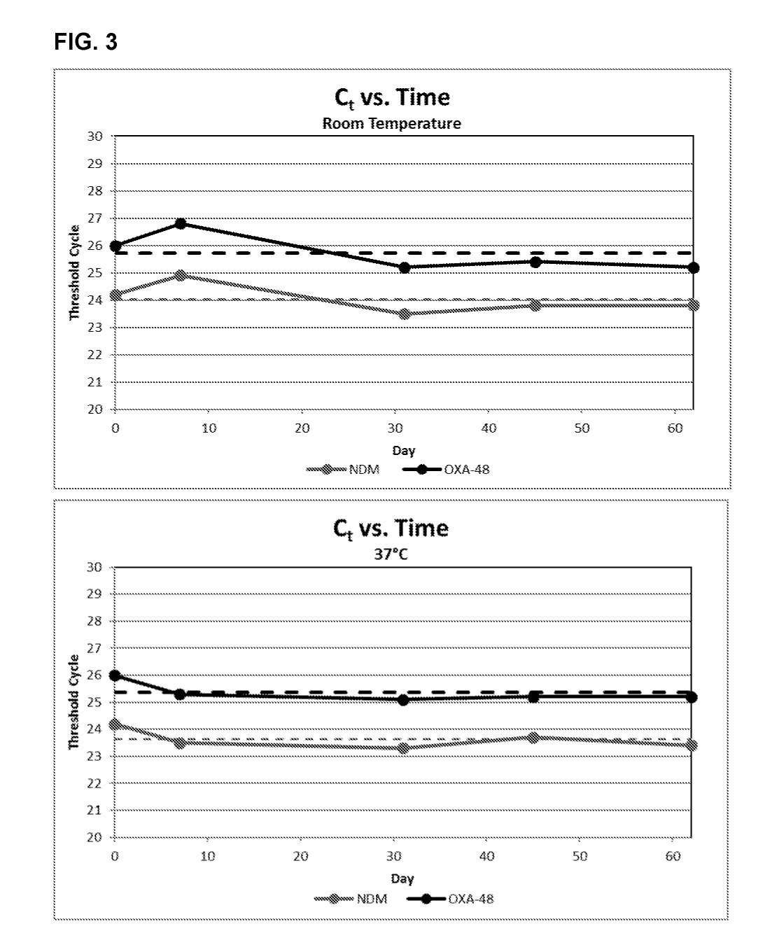

Also, following testing at the 120 day time point, aliquots of the control were transferred to storage at room temperature and 37° C.; these samples were analyzed following 7, 31, 45, and 62 days of storage at the elevated temperatures and no significant change in the determined Ct values was observed as shown in FIG. 3. FIG. 3 depicts the determined threshold cycle of four suspensions of K. pneumoniae with NDM and OXA-48 genes at 1×10⁷ cells/m L. The mean value of all measurements at a given concentration is represented by the dashed line.

### Example 3

Prepared controls of the present teachings were evaluated with the BioFire FilmArray BCID. Klebsiella pneumoniae containing a KPC resistance gene and Escherichia coli were used to prepare a simulated positive blood culture sample by diluting stabilized E. coli cells to 3.5×10⁴ cells/mL and stabilized K. pneumoniae cells to 2×10⁶ cells/mL in a solution comprised of 3 mL BD BACTEC Aerobic/F Blood Culture Media (Becton Dickinson and Company) and 0.8 mL of stabilized blood (Streck, Inc). Simulated positive blood culture samples were stored at 4° C. and analyzed at day 0, day 14, day 30, day 45, day 72, day 90, day 120 day 150, and day 180. The stabilized bacteria and resistance mechanism were detected and there was no change in detected positive samples over the course of 180 days. The results demonstrate the stability of simulated positive blood culture for up to 180 days at 4° C. Also, following testing at the 120 day time point, aliquots of the control were transferred to storage at room temperature and 37° C.; these samples were analyzed following 32 days of storage at the elevated temperatures and no change in the detected positive samples was observed.

The present teachings demonstrate that cells containing target gene sequences can be stabilized and subsequently used in PCR and RT-PCR based applications. The present teachings provide molecular reference controls which mimic a true patient sample with an external positive control containing one or more cell types stabilized at a known concentration (cells/mL). The stabilized cells contain gene targets corresponding to specific molecular diagnostic tests and are suspended in a matrix similar to those used in molecular tests used for the screening and diagnosing diseases in clinical laboratories. The present teachings provide molecular reference controls which are carried through all sample preparation, extraction, and amplification steps. The present teachings provide molecular reference controls with a superior storage and handling solution for customers as compared to current controls.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of, the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. Likewise, any reference to “first” or “second” items is not intended to foreclose additional items (e.g., third, fourth, or more items); such additional items are also contemplated, unless otherwise stated.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints. The specification of ranges herein also contemplates individual amounts falling within the range. Thus, for example, a range of 10 to 15 contemplates individually the amounts of 10, 11, 12, 13, 14, and 15.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

1. A control composition for indicating positive presence of a condition comprising:
at least one biological fluid or biological fluid component;
at least one condition component processed to be indicative of a condition; and
at least one cross-linking agent.

2. The control composition of claim 1, wherein the control composition includes an aldehyde and/or aldehyde donor agent.

3. The control composition of claim of any of the preceding claims, wherein the at least one biological fluid is blood.

4. The control composition of any of the preceding claims, wherein the at least one biological fluid is selected from the group consisting of blood, serum, plasma, urine, fecal matter, saliva, sputum, cerebral spinal fluid, vaginal secretions, and semen.

5. The control composition of any of the preceding claims, wherein the at least one biological fluid is blood culture or bacterial culture.

6. The control composition of any of the preceding claims, wherein the at least one biological fluid or biological fluid component is of human origin.

7. The control composition of any of the preceding claims, wherein the at least one biological fluid or biological fluid component is of animal origin.

8. The control composition of any of the preceding claims, wherein the at least one biological fluid component is selected from the group consisting of white blood cells, red blood cells, cell-free nucleic acids, proteins, fragmented nucleic acids, fragmented proteins, and any synthetic or engineered combination thereof.

9. The control composition of any of the preceding claims, wherein the at least one biological fluid component is a nucleic acid or protein.

10. The control composition of any of the preceding claims, wherein the at least one condition component indicative of a condition is selected from the group consisting of inactivated viruses, attenuated viruses, plasm ids, bacteria, fungi, parasites, microbiota, engineered cell lines, wild-type cells and/or any microbiome combination thereof.

11. The control composition of any of the preceding claims, wherein the at least one biological fluid component is an endogenously and/or exogenously modified component to indicate one or more of mutation, glycosylation, methylation, acetylation, ubiquitination, S-Nitrosylation, lipidation, GPI anchors, myristoylation, palmitoylation, prenylation, and phosphorylation.

12. The control composition of any of the preceding claims, wherein the at least one condition component is an inactivated pathogen.

13. The control composition of any of the preceding claims, wherein the at least one condition component is an inactivated virus.

14. The control composition of any of the preceding claims, wherein the at least one condition component is bacteria.

15. The control composition of any of the preceding claims, wherein the at least one condition component is at least one Gram-negative organism that contains at least one mechanism of drug or treatment resistance.

16. The control composition of any of the preceding claims, wherein the at least one condition component is at least one Gram-positive organism that contains at least one mechanism of drug or treatment resistance.

17. The control composition of any of the preceding claims, wherein the at least one condition component includes an immortalized and/or primary cell line containing a mutant phenotype or other molecular signature of interest.

18. The control composition of any of the preceding claims, wherein the control composition serves an external control.

19. The control composition of any of the preceding claims, wherein the control composition serves as an internal control.

20. The control composition of any of the preceding claims, wherein the control composition serves as an internal control and the at least one biological fluid component is a nucleic acid that is a control target of interest.

21. The control composition of any of the preceding claims, wherein the at least one biological fluid component is treated to mimic effects of a condition.

22. The control composition of any of the preceding claims, wherein the at least one biological fluid is substantially free of any treatment to mimic effects of a condition.

23. The control composition of any of the preceding claims, wherein the control composition is substantially free of any time-dependent component degradation when stored at room temperature.

24. The control composition of any of the preceding claims, wherein the control composition is substantially free of any time-dependent component degradation when stored at 4° C.

25. The control composition of any of the preceding claims, wherein the control composition is substantially free of any time-dependent component degradation when stored at 37° C.

26. The control composition of any of the preceding claims, wherein the at least one cross-linking agent includes an aldehyde with one or more reactive groups.

27. The control composition of any of the preceding claims, wherein the at least one cross-linking agent includes formaldehyde, glutaraldehyde, phthaldehyde or a combination thereof.

28. The control composition of any of the preceding claims, wherein the at least one cross-linking agent includes N-Hydroxysuccinimide (NHS) ester functional group(s).

29. The control composition of any of the preceding claims, wherein the at least one cross-linking agent includes disuccinim idyl suberate and/or bis[sulfosuccinim idyl] suberate.

30. The control composition of any of the preceding claims, wherein the at least one cross-linking agent includes maleimide functional group(s).

31. The control composition of any of the preceding claims, wherein the at least one cross-linking agent includes dithiobismaleimidoethane (DTME) and/or bismaleimidohexane (BMH).

32. The control composition of any of the preceding claims, wherein the at least one cross-linking agent includes one or more functional groups of the following: haloacetyls, imidoesters, pyridyl disulfides, hydrazides, alkoxyamines, and carboiimides.

33. The control composition of any of the preceding claims, wherein the at least one cross-linking agent is homobifunctional.

34. The control composition of any of the preceding claims, wherein the at least one cross-linking agent is heterobifunctional.

35. The control composition of any of the preceding claims, wherein the control composition includes a formaldehyde donor agent including imidazolidinyl urea (IDU), diazolidinyl urea (DU) or a combination thereof.

36. The control composition of any of the preceding claims, wherein the control composition includes chemical analytes and/or metabolites.

37. The control composition of any of the preceding claims, wherein the control composition includes one or more surfactants.

38. The control composition of any of the preceding claims, wherein the control composition includes one or more metabolic inhibitors.

39. The control composition of any of the preceding claims, wherein the control composition includes one or more nuclease inhibitors.

40. The control composition of any of the preceding claims, wherein the control composition includes one or more organic components.

41. The control composition of any of the preceding claims, wherein the control composition is utilized for one or more of the following: nucleic acid/protein purification protocols, sample-to-answer testing platforms, polymerase chain reaction protocols, fluorescence in situ hybridization (FISH) testing, genetic sequencing, flow cytometry, enzymatic testing, mass spectrometry or enzyme-linked immunosorbent assay (ELISA) testing.

42. The control composition of any of the preceding claims, wherein the control composition includes a stabilization period of about 15 minutes to about 120 hours.

43. The control composition of any of the preceding claims, wherein the control composition includes a stabilization period of about 15 minutes.

44. The control composition of any of the preceding claims, wherein the control composition is stable at room temperature.

45. The control composition of any of the preceding claims, wherein the control composition is stable for at least up to 180 days at one or more of the following: 4° C., room temperature and 37° C.
